(19) United States
(12) Patent Application Publication
(10) Pub. No.: US 2015/0080657 A1
(43) Pub. Date: Mar. 19, 2015

(54) ENDOSCOPE FOGGING PREVENTION UNIT AND ENDOSCOPE

(71) Applicant: OLYMPUS CORPORATION, Tokyo (JP)

(72) Inventor: Takayuki IDE, Akishima-shi (JP)

(73) Assignee: OLYMPUS CORPORATION, Tokyo (JP)

(21) Appl. No.: 14/263,211

(22) Filed: Apr. 28, 2014

(30) Foreign Application Priority Data
Sep. 18, 2013 (JP) 2013-193556

(51) Int. Cl. A61B 1/12

(52) U.S. Cl. CPC A61B 1/127
USPC 600/117

(57) ABSTRACT

An endoscope fogging prevention unit includes: a distal end section including a cylindrical member, in an interior of which at least an optical member is provided; a heating section configured to heat at least the optical member; and a measuring section configured to measure a temperature of the interior of the distal end section. The fogging prevention unit further includes: a wiring substrate section including a mounting area portion and a wiring area portion; and a conducting section having the lowest insulating properties against the outside in the mounting area portion, and connected to a ground of a system different from a system of a wiring member of the heating section and the measuring section.

### CROSS-REFERENCE TO RELATED APPLICATIONS

This application is based upon and claims the benefit of priority from prior Japanese Patent Application No. 2013-193556, filed Sep. 18, 2013, the entire contents of which are incorporated herein by reference.

### BACKGROUND

1. Field of the Invention

The present invention relates to an endoscope fogging prevention unit and an endoscope for preventing fogging that occurs in the endoscope.

2. Description of the Related Art

An insertion section of an endoscope is inserted into a highly humid environment, such as a cavity of the body. When the insertion section is inserted into such the environment and the temperature of the insertion section that has been inserted is lower than the temperature of the environment, fogging may be occur on a surface of an optical member (such as a lens cover) provided at a distal end section of the insertion section because of a difference in temperature. There is the possibility that such fogging may interfere with observation or treatment.

In order to deal with the fogging, an endoscope is configured to include a fogging prevention unit provided inside a distal end section of an insertion section and configured to prevent fogging. The fogging prevention unit prevents fogging by heating the interior of the distal end section including an optical member. For that purpose, the fogging prevention unit includes a heater for heating the interior, a temperature sensor for measuring a temperature of the interior, and a substrate on which the heater and the temperature sensor are mounted.

An example of the above-described fogging prevention unit is disclosed is Jpn. Pat. Appln. KOKAI Publication No. 2006-282. Jpn. Pat. Appln. KOKAI Publication No. 2006-282 discloses controlling heating by a heater on the basis of a temperature detected by a temperature sensor.

### BRIEF SUMMARY OF THE INVENTION

An aspect of an endoscope fogging prevention unit of the present invention includes: a distal end section including a cylindrical member, in an interior of which at least an optical member is provided; a heating section provided in the interior of the distal end section and configured to heat at least the optical member; a measuring section provided in the interior of the distal end section and configured to measure a temperature of the interior of the distal end section; a wiring substrate section including a mounting area portion in which the heating section and the measuring section are mounted, and a wiring area portion in which a wiring member connected to the heating section and the measuring section is provided; and a conducting section provided in the mounting area portion, having the lowest insulating properties against the outside in the mounting area portion, and connected to a ground of a system different from a system of the wiring member of the heating section and the measuring section.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 illustrates an interior configuration of a distal end section of an insertion section of an endoscope according to the present invention.

FIG. 2 illustrates a configuration of a heating unit.

FIG. 3 illustrates Configurations 1 and 2 of an endoscope fogging prevention system according to a first embodiment.

FIG. 4A is a top view of the heating unit.

FIG. 4B is a cross-sectional view cut along line 4B-4B of FIG. 4A.

FIG. 4C is a cross-sectional view cut along line 4C-4C of FIG. 4A.

FIG. 4D is a cross-sectional view cut along line 4D-4D of FIG. 4A.

FIG. 5A is a cross-sectional view cut along line 4C-4C, in which a first sealing member is provided in the state shown in FIG. 4A.

FIG. 5B is a cross-sectional view cut along line 4D-4D, in which a first sealing member is provided in the state shown in FIG. 4A.

FIG. 6A is a cross-sectional view cut along line 4C-4C, in which a second sealing member is provided in the state shown in FIG. 5A, according to a second embodiment.

FIG. 6B is a cross-sectional view cut along line 4D-4D, in which a second sealing member is provided in the state shown in FIG. 5A, according to the second embodiment.

### DETAILED DESCRIPTION

Hereinafter, a detailed description is given on the embodiments of the present invention with reference to the accompanying drawings.

### First Embodiment

[Configuration]

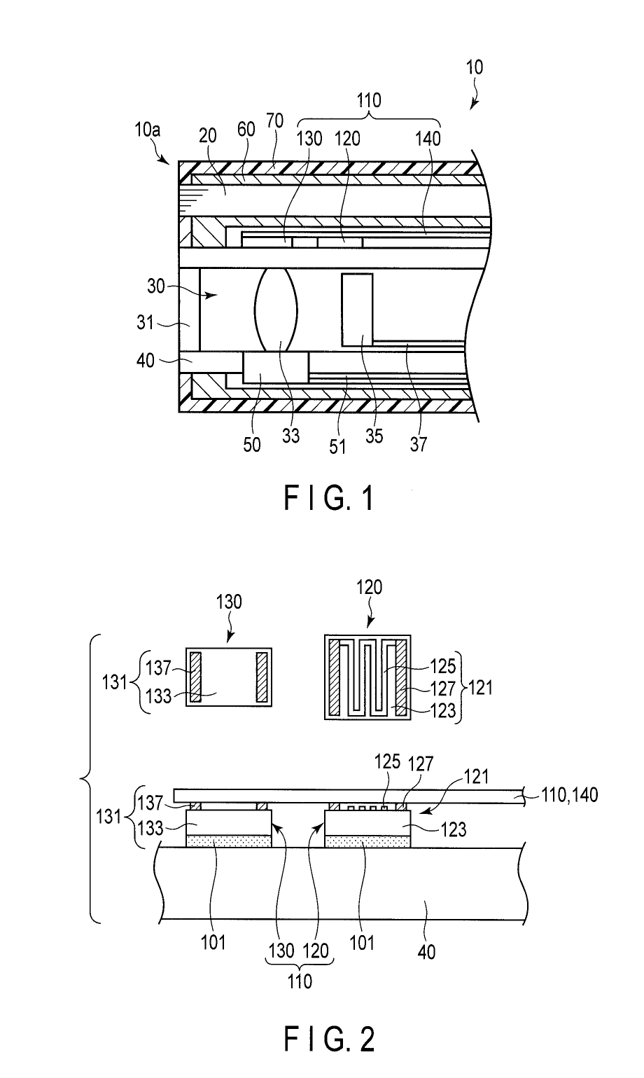

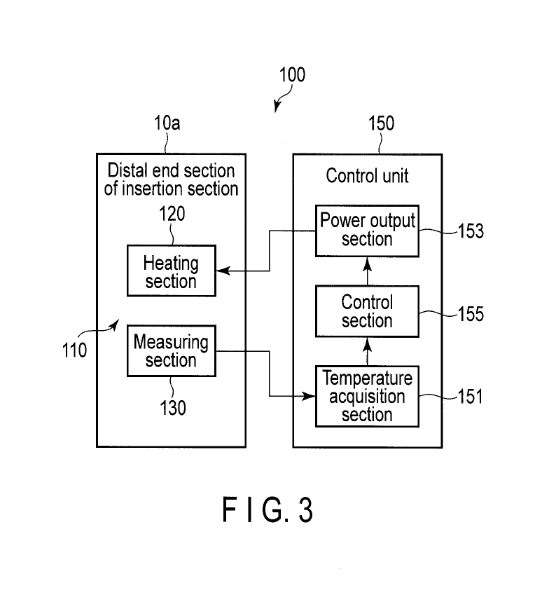

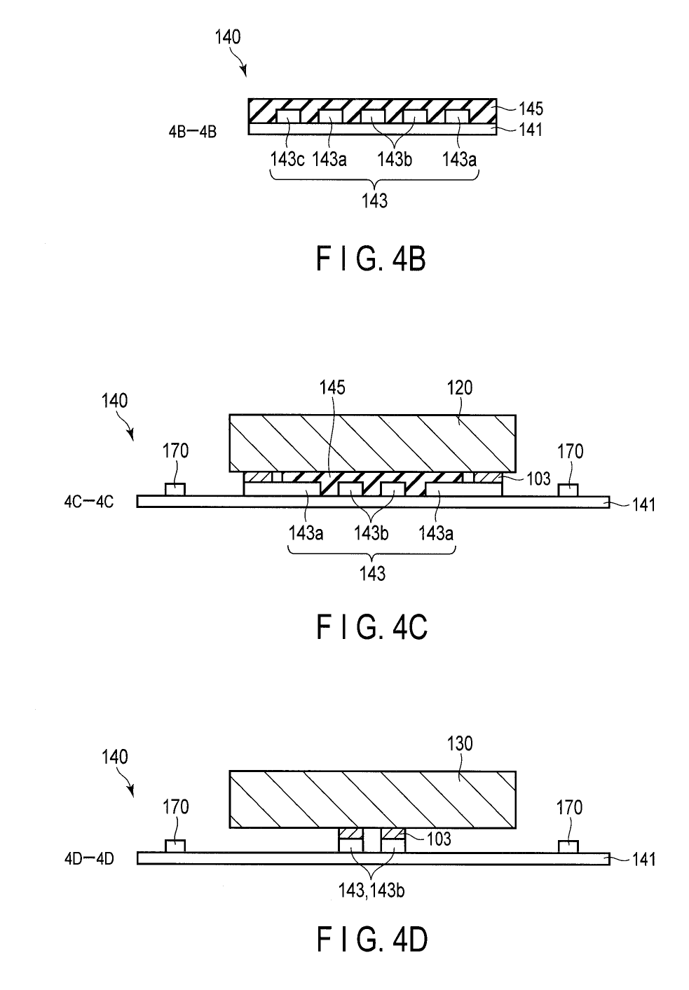

A first embodiment will be described with reference to FIGS. 1, 2, 3, 4A, 4B, 4C, 4D, 5A, and 5B. For the sake of clarity of illustration, some members are omitted in some of the drawings; for example, a bonding material 103 and a conducting section 170 are omitted in FIG. 2, and a first sealing member 171 is omitted in FIG. 4A.

[Configuration of Distal End Section 10a of Endoscope]

As shown in FIG. 1, an endoscope, not shown, includes a hollow, elongated insertion section 10, which is inserted into a lumen such as in a body cavity. The distal end section 10a of the insertion section 10 includes a light guide 20, which illuminates an observation target with illumination light by guiding the illumination light thereto, and an image pickup unit 30 configured to capture an image of the observation target. The distal end section 10a further includes a lens frame 40 holding the image pickup unit 30, and a drive element 50 provided in the lens frame 40 and configured to focus and zoom by driving the lens 33 of the image pickup unit 30.

Since the light guide 20 is connected with a light source device, not shown, via the insertion section 10 and an operating section, not shown, of the endoscope, light is supplied to the light guide 20. The light guide 20 emits illumination light toward the outside from a distal end section of the light guide 20.

The image pickup unit 30 includes a lens cover 31 provided inside the distal end section 10a so as to be exposed from a distal end surface of the distal end section 10a toward the outside, and a lens 33 provided at the back of the lens cover 31. The image pickup unit 30 further comprises an image pickup device 35 provided at the back of the lens 33, and an image pickup cable 37, which is connected with the image pickup device 35, and via which electricity is supplied to the image pickup device 35, a control signal for controlling the image pickup device 35 is sent to the image pickup device 35, and a video signal captured by the image pickup device 35 is transmitted.

The image pickup cable 37 is inserted up through a connection connector via the insertion section 10, the operating section, and a universal cord. Since the connection connector is connected with a control device, not shown, for controlling the endoscope, the image pickup cable 37 is connected with the control device. Thereby, the electricity that drives the image pickup device 35 and the control signal are supplied to the image pickup cable 37. Thus the image pickup cable 37 supplies and sends the electricity and the control signal to the image pickup device 35. Since the connection connector is connected with the control device, a video signal captured by the image pickup device 35 is transmitted to the control device.

The lens cover 31 does not need to be a cover member in a simple plate shape, but may be formed in the shape of a lens. In the description that follows, at least one of the lens cover 31 and the lens 33 of the distal end section 10a, which prevent fogging when the insertion section 10 is inserted into a body cavity or the like, will be referred to as an optical member. The optical member only needs to be provided inside the distal end section 10a, so as to be exposed toward the outside from the distal end surface of the distal end section 10a, for example.

The drive element 50 includes a motor, for example. The drive element 50 is connected to a drive cable 51, via which electricity is supplied to the drive element 50 and a drive signal for controlling the drive element 50 is sent to the drive element 50.

The drive cable 51 is inserted up through the connection connector via the insertion section 10, the operating section, and the universal cord. Since the connection connector is connected with the control device, not shown, the drive cable 51 is connected with the control device. Thereby, the electricity and the control signal that drive the drive element 50 are supplied to the drive cable 51. The drive cable 51 supplies the electricity and the control signal to the drive element 50.

The lens frame 40 is formed of a cylindrical member, for example. The lens frame 40 houses the image pickup unit 30 in the cylinder.

As shown in FIG. 1, the distal end section 10a further includes an inner frame 60 holding the light guide 20 and the lens frame 40, and an outer frame 70 covering the inner frame 60 and functioning as the outermost layer of the distal end section 10a.

The inner frame 60 is formed of metal, for example, and the outer frame 70 is formed of a resin, for example.

[Fogging of Optical Member]

The endoscope including the distal end section 10a is usually provided in an environment in which the temperature and humidity are managed, such as a treatment room. Accordingly, the distal end section 10a is subjected to such a temperature and humidity before use. When the insertion section 10 is inserted into a body cavity, a difference in temperature between the room and the body or a highly humid environment (humidity of approximately 98%-100%) of the interior of the body cavity, for example, causes fogging in the optical member such as the lens cover 31, which significantly reduces the imaging field of vision.

[Configuration 1 of Endoscope Fogging Prevention System 100 (Prevention Unit 100)]

In view of the above, in the endoscope and the control device, not shown, for controlling the endoscope, an endoscope fogging prevention system 100 configured to prevent fogging of the endoscope is provided, as shown in FIGS. 1, 2, and 3. The fogging prevention system 100 includes an endoscope fogging prevention unit (hereinafter referred to as a prevention unit 110) provided inside the distal end section 10a of the insertion section 10 and configured to prevent fogging that occurs in the optical member provided inside the distal end section 10a.

The prevention unit 110 includes a heating section 120 provided in the lens frame 40, for example, and including a heater configured to heat the interior of the distal end section 10a including the lens cover 31 via the lens frame 40 so as to prevent fogging that occurs in the optical member such as the lens cover 31, and a measuring section 130 provided in the lens frame 40, for example, and including a temperature sensor configured to measure the temperature of the interior of the distal end section 10a including the lens cover 31 via the lens frame 40. The prevention unit 110 further includes a wiring substrate section 140, which is a flexible substrate on which the heating section 120 and the measuring section 130 are mounted by means of a surface mounting technique, for example.

As shown in FIG. 2, the back surface of the prevention unit 110, for example, is bonded to an outer periphery of the lens frame 40, for example, via an adhesive agent 101 having, for example, a high thermal conductivity. Alternatively, a very thin layer of an adhesive agent having a low conductivity may be applied as the adhesive agent 101. As shown in FIG. 1, the heating section 120 and the measuring section 130 only need to be provided inside the distal end section 10a. Accordingly, the heating section 120 and the measuring section 130 may be provided in an inner frame 60 holding the lens unit, for example. The lens unit includes the lens cover 31, the lens 33, and the lens frame 40 holding the lens cover 31 and the lens 33, for example.

As shown in FIGS. 1 and 2, the heating section 120 is provided so as to be adjacent to the measuring section 130 in a longitudinal direction of the distal end section 10a, for example. The heating section 120 is provided at a desired interval from the measuring section 130, for example. The heating section 120 is arranged at a distance farther from the lens cover 31 (surface of the distal end section 10a) than the measuring section 130, for example. It is to be noted that the placement of the heating section 120 and the measuring section 130 may be reversed. Further, the positional relationship between the heating section 120 and the measuring section 130 is not particularly limited. Moreover, the heating section 120 and the measuring section 130 may be mounted on different flexible substrates.

[Heating Section 120]

The heating section 120 heats the interior of the distal end section 10a such that the temperature of the lens cover 31 becomes higher than the temperature of the body but not so high as to damage the body tissue, for example. The temperature is set in a range between approximately 38° C. and 42° C., for example. The heating section 120 heats the interior of the distal end section 10a such that the temperature of the optical member is set within the above-described range. The heating section 120 may either directly heat the optical member, or indirectly heat the optical member via the lens frame 40 or the inner frame 60, for example.

As shown in FIG. 2, the heating section 120 includes a heating chip 121, for example. The heating chip 121 includes a ceramic substrate 123, a metal resistance 125 provided on the substrate 123, and a pad 127 provided on the substrate 123 and electrically connected to the metal resistance 125. The metal resistance 125 is formed in the shape of a thin film or paste, and functions as a heating element. The pad 127 is formed as a current introduction terminal. The heating pad 121 may be formed as a resistive element formed of a bulk obtained by molding a resistive material in the shape of chips by sintering, for example. In the description that follows, the term “bulk” refers to chip-shaped molding obtained by sintering a material, for example.

[Measuring section 130]

The measuring section 130 measures the temperature of the interior of the distal end section 10a, for example.

As shown in FIG. 2, the measuring section 130 includes a temperature sensor chip 131, for example. The temperature sensor chip 131 includes a thermistor body 133 formed of a bulk, and a pad 137 provided on the thermistor body 133 and electrically connected to the thermistor body 133. The thermistor body 133 functions as a temperature-sensing element. The pad 137 is formed as a current introduction terminal. It is also possible to make a ceramic substrate a base of the temperature sensor chip 131, as in the heating chip 121, and form a thermistor resistance or a metal resistance in the shape of a thin film or paste on the ceramic substrate.

[Wiring Substrate Section 140]

As shown in FIGS. 4A, 4B, 4C, and 4D, the heating section 120 and the measuring section 130 are mounted on the wiring substrate section 140. Accordingly, the wiring substrate section 140 includes a base layer 141, a wiring portion 143 provided on the base layer 141 and connected to the heating section 120 and the measuring section 130, and a cover member 145 covering the wiring portion 143 and having insulating properties.

As shown in FIG. 4A, the wiring substrate section 140 includes a wiring area portion 140a, which functions as an area portion in which the wiring portion 143 connected to the heating section 120 and the measuring section 130 is mainly mounted, and a mounting area portion 140b, which functions as an area portion in which the heating section 120 and the measuring section 130 are mainly mounted.

The base layer 141 includes an insulating film formed of polyimide, for example.

The wiring portion 143 includes a heating-side wiring portion 143a connected to the heating section 120, and a measuring-side wiring portion 143b connected to the measuring section 130. The heating-side wiring portion 143a and the measuring-side wiring portion 143b are provided on the base layer 141. The heating-side wiring portion 143a and the measuring-side wiring portion 143b are of different systems. The heating-side wiring portion 143a and the measuring-side wiring portion 143b are formed of copper foil.

As shown in FIGS. 4A and 4C, one end portion of the heating-side wiring portion 143a is bonded to the pad 127 (not shown in FIGS. 4A and 4C) of the heating section 120 via a bonding material 103, which may be solder, for example. Thereby, the heating section 120 is electrically connected to the heating-side wiring portion 143a. The one end portion including the bonding material 103 and the pad 127 function as an electrical connection part between the heating section 120 and the heating-side wiring portion 143a.

Further, as shown in FIGS. 4A and 4D, one end portion of the measuring-side wiring portion 143b is bonded to the pad 137 (not shown in FIGS. 4A and 4C) of the measuring section 130 via a bonding material 103, which may be solder, for example. Thereby, the measuring section 130 is electrically connected to the measuring-side wiring portion 143b. The one end portion including the bonding material 103 and the pad 137 function as an electrical connection part between the measuring section 130 and the measuring-side wiring portion 143b.

The other end portion of the heating-side wiring portion 143a and the other end portion of the measuring-side wiring portion 143b function as a lead portion exposed from a cover member 145, as shown in FIG. 4A. The other end portions of the heating-side wiring portion 143a and the measuring-side wiring portion 143b are connected to a lead line, not shown. The lead line is inserted up through the connection connector via the insertion section 10, the operating section, and the universal cord. Since the connection connector is connected with the control device, the wiring portion 143 is connected with the control device. Thereby, the electricity and the control signal that drive the heating section 120 are supplied to the heating section 120 via the lead line and the heating-side wiring portion 143a. Further, the electricity and the control signal that drive the measuring section 130 are supplied to the measuring section 130 via the lead line and the measuring-side wiring portion 143b. Since the connection connector is connected with the control device, temperature data contained in detection data detected by the measuring section 130 is transmitted to the control device via the measuring-side wiring portion 143b and the lead line.

As shown in FIGS. 4A and 4B, the cover member 145 includes an insulating film formed of polyimide, for example. The cover member 145 covers the entire wiring portion 143 including the base layer 141 in the wiring area portion 140a. The cover member 145 covers the heating-side wiring portion 143a and the measuring-side wiring portion 143b excluding the electrical connection part between the heating section 120 and the measuring section 130 in the mounting area portion 140b, as shown in FIGS. 4A, 4C, and 4D.

[Configuration 2 of Fogging Prevention System 100 of Endoscope (Control Unit 150)]

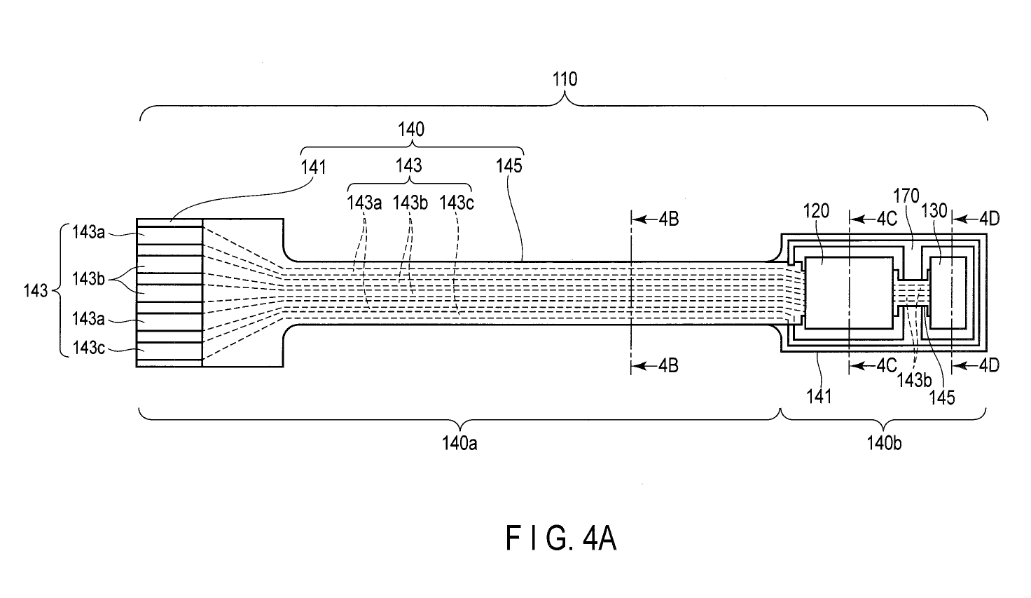

The fogging prevention system 100 further includes a control unit 150 configured to control driving of the heating section 120 on the basis of temperature information of the interior of the distal end section 10a measured by the measuring section 130, as shown in FIG. 3. The control unit 150 may, for example, be formed separately from the endoscope. The control unit 150 is provided in the control device, not shown, connected to the universal cord of the endoscope, for example, and configured to control the endoscope.

As shown in FIG. 3, the control unit 150 includes a temperature acquisition section 151 configured to acquire the actual temperature of the interior of the distal end section 10a measured by the measuring section 130, and a power output section 153 configured to output to the heating section 120 an amount of electricity (hereinafter referred to as heater driving power) necessary for driving the heating section 120.

As also shown in FIG. 3, the control unit 150 further includes a control section 155 configured to control the power output section 153, such that the control section 155 calculates a difference between a temperature acquired by the temperature acquisition section 151 and a target temperature set in advance, and calculates an amount of heater driving power that eliminates the difference on the basis of the calculated difference, and the power output section 153 outputs the calculated amount of heater driving power to the heating section 120. The target temperature includes a temperature that prevents fogging of an optical member such as the lens cover 31 by heating the optical member, for example. Further, the target temperature includes a temperature in the outer frame 70, which is the outermost part of the distal end section 10a, and in particular, is a temperature in the vicinity of the heating section 120 that is at or below a temperature that is not too high to damage body tissue. The target temperature can, for example, be appropriately adjusted as desired by the control unit 150, for example. The target temperature is recorded in advance in a recording section, not shown, provided in the control unit 150, for example.

The temperature acquisition section 151 acquires a temperature at a desired timing or during a desired period of time, for example.

A feedback on the temperature measured by the measuring section 130 is given to the control unit 150. By repeatedly giving feedback, the temperature of the interior of the distal end section 10a is controlled with high precision such that the heating temperature of the heating section 120 is set to the target temperature. Examples of approaches for controlling the heating section 120 include ON-OFF control, PWM control, and PID control.

[Insulating Properties of Prevention Unit 110]

In the above-described feedback, the insulating properties between the prevention unit 110 and the interior of the distal end section 10a play an important factor in controlling the temperature of the interior of the distal end section 10a with high precision.

When a surgical endoscope is used together with a treatment tool, not shown, for treating an affected part by an electric action, for example, there is the possibility that the prevention unit 110 may be effected by static electricity, for example, from the outside of the prevention unit 110, such as the treatment tool. Static electricity may cause a deterioration in performance of the prevention unit 110, such as the temperature control performance. It is therefore necessary for the prevention unit 110 to have increased resistance to static electricity and have insulating properties.

[Conducting Section 170 and Conductor-side Wiring Portion 143c]

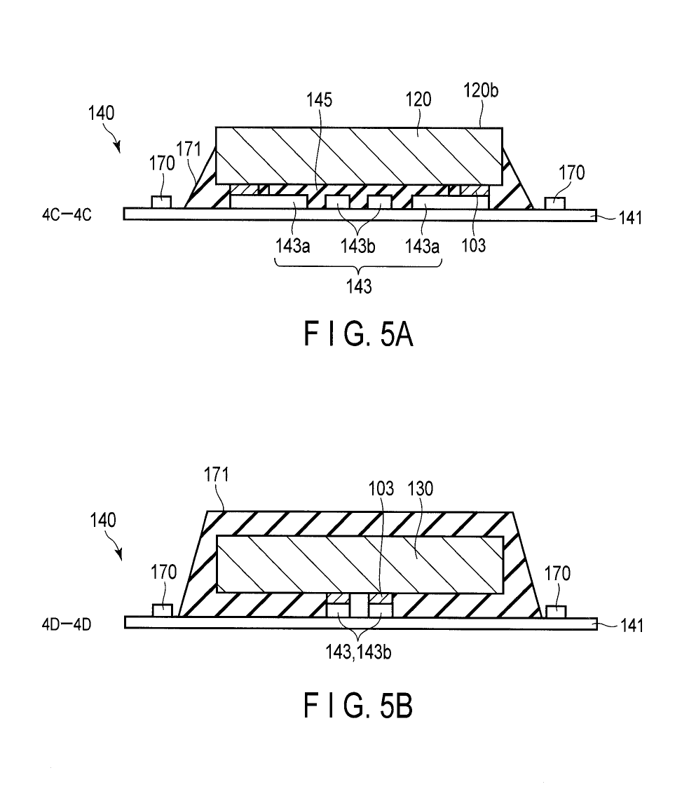

As shown in FIGS. 4A, 4C, 4D, 5A, and 5B, in consideration of the above-described insulating properties, the prevention unit 110 further includes a conducting section 170 provided on the base layer 141 so as to surround the heating section 120 and the measuring section 130 in the plane direction of the base layer 141 and be exposed to the cover member 145.

As shown in FIG. 4A, the conducting section 170 is provided only in the mounting area portion 140b. The conducting section 170 is formed approximately in the shape of a frame in a figure of the numeral eight (8), for example. The conducting section 170 is provided in the periphery of the heating section 120 and in the periphery of the measuring section 130. Further, the conducting section 170 is provided in the periphery of the heating section 120 and the periphery of the measuring section 130 in the plane direction of the mounting area portion 140b, so as to surround the heating section 120 and the measuring section 130. The conducting section 170 surrounding the heating section 120 is integrally formed with the conducting section 170 surrounding the measuring section 130. The conducting section 170 is provided in proximity to the heating section 120 and the measuring section 130.

Further, as shown in FIG. 4A, the wiring portion 143 further includes a conductor-side wiring portion 143c electrically connected to the conducting section 170. The conductor-side wiring portion 143c is of a system different from that of the heating-side wiring portion 143a and the measuring-side wiring portion 143b. The conductor-side wiring portion 143c and the conducting section 170 are formed of a series of copper foil. As shown in FIG. 4B, the conductor-side wiring portion 143c is covered by one cover member 145, together with the heating-side wiring portion 143a and the measuring-side wiring portion 143b. The conductor-side wiring portion 143c should preferably have a width greater than that of the heating-side wiring portion 143a and the measuring-side wiring portion 143b.

As shown in FIG. 4A, one end portion of the conductor-side wiring portion 143c is placed in an interface region between the wiring area portion 140a and the mounting area portion 140b, and is integrally formed with the conducting section 170 in the interface region. The other end portion of the conductor-side wiring portion 143c is connected to a ground (GND) provided in the control device, not shown, via a lead line, not shown. The GND is of a system different from that of the heating-side wiring portion 143a and the measuring-side wiring portion 143b.

Thereby, the conducting section 170 is connected to the GND via the conductor-side wiring portion 143c and the lead line.

In the present embodiment, it is necessary to prevent a signal from accidentally flowing to the conducting section 170, the conductor-side wiring portion 143c, and the lead line. It is therefore necessary for the conducting section 170 to have desired insulating resistance to the outside. In other words, the conducting section 170 has the lowest insulating properties against the outside in the mounting area portion 140b. In view of the above, Configurations 1-3, as will be listed below, can be taken as exemplary configurations.

Configuration 1: The conducting section 170 includes a non-conducting section provided in an outer periphery of the conducting section 170.

Configuration 2: A space is provided between the conducting section 170 and the endoscope structure.

Configuration 3: Resistance elements such as a resistive element, a capacitor element, a varistor element, and a diode element are provided between the conducting section 170 and the GND.

[First Sealing Member 171]

As shown in FIGS. 5A and 5B, in consideration of the insulating properties, the prevention unit 110 further includes a first sealing member 171 provided at least in the connection part between the wiring portion 143 and the heating section 120 and the measuring section 130 such that the conducting section 170 is exposed, configured to seal the connection part, and having insulating properties. The first sealing member 171 is omitted in drawings other than FIGS. 5A and 5B in the present embodiment, for the sake of clarity of illustration. The first sealing member 171 insulates electricity and a control signal to be supplied to the heating section 120 and the measuring section 130 from the outside.

The first sealing member 171 is provided only in the mounting area portion 140b. The first sealing member 171 is formed in the shape of a frame. Further, the first sealing member 171 is provided in the periphery of the heating section 120 so as to surround the heating section 120 in the plane direction of the base layer 141, in an approximately same manner as in the conducting section 170. Further, the first sealing member 171 is provided in the periphery of the measuring section 130 so as to surround the measuring section 130 in an approximately same manner as in the conducting section 170.

As shown in FIG. 5A, the connection part between the wiring portion 143 (heating-side wiring portion 143a) and the heating section 120 includes a surface of the base layer 141, the periphery of the heating-side wiring portion 143a, the periphery of the bonding material 103, and the periphery of the heating section 120, for example. The connection part includes an electrical connection part between the heating section 120 and the heating-side wiring portion 143a. The first sealing member 171 is provided between the conducting section 170 and the heating-side wiring portion 143a in the plane direction of the base layer 141.

When, for example the heating section 120 includes the heating chip 121, the substrate 123, the metal resistance 125, and the pad 127, the ceramic substrate 123 has insulating properties. Accordingly, in consideration of heat conducting properties, the first sealing member 171 seals the connection part between the wiring portion 143 and the heating section 120 such that a bonding surface 120b of the heating section 120 bonded to the lens frame 40 is exposed, as shown in FIG. 5A.

As shown in FIG. 5B, the connection part between the wiring portion 143 (measuring-side wiring portion 143b) and the measuring section 130 includes a surface of the base layer 141, the periphery of the measuring-side wiring portion 143b, the periphery of the bonding material 103, and the periphery of the measuring section 130, for example. The connection part includes an electric connection part between the measuring section 130 and the measuring-side wiring portion 143b. The first sealing member 171 is provided between the conducting section 170 and the measuring-side wiring portion 143b in the plane direction of the base layer 141.

When the measuring section 130 is formed of a bulk, for example, the first sealing member 171 seals the entire heating section 120 including the connection part, such that the conducting section 170 is exposed and the measuring section 130 is embedded in the first sealing member, as shown in FIG. 5B.

As a result thereof, the conducting section 170 has the lowest insulating properties against the outside in the mounting area portion 140b.

[Operation Method]

An operation method of the present embodiment will now be discussed.

Static electricity applied from the outside enters into the base layer 141, the wiring portion 143, the connection part between the heating-side wiring portion 143a and the heating section 120, the connection part between the measuring-side wiring portion 143b and the measuring section 130, the heating section 120, and the measuring section 130. As a result thereof, the heating section 120 and the measuring section 130 may be denaturized, or may be damaged. In such a case, there is the possibility that the prevention unit 110 cannot maintain desired performance.

In the present embodiment, on the other hand, the base layer 141 has insulating properties, and the wiring portion 143 is covered with the cover member 145 having insulating properties. This prevents entry of static electricity into the base layer 141 and the wiring portion 143.

Further, by setting the base layer 141 and the cover member 145 to have a desired thickness and setting a margin from the wiring portion 143 to an end portion of the wiring substrate section 140 as desired, entry of static electricity into the base layer 141 and the wiring portion 143 is prevented.

In the present embodiment, the connection part between the heating-side wiring portion 143a and the heating section 120, the connection part between the measuring-side wiring portion 143b and the measuring section 130, the heating section 120, and the measuring section 130 are sealed by the first sealing member 171 having insulating properties. This prevents entry of static electricity into the sealed area.

There is the possibility, however, that static electricity enters from an area having low insulating properties. The area having low insulating properties includes, for example, an interface between the heating section 120 and the first sealing member 171, an interface between the measuring section 130 and the first sealing member 171, an interface between the base layer 141 and the first sealing member 171, and a part of the first sealing member 171 having a small thickness. That is, the area having low insulating properties corresponds to the mounting area portion 140b.

A common approach to prevent the above-described entry of static electricity is to increase a creepage distance in which static electricity enters, i.e., to cause the first sealing member 171 to firmly perform the sealing. In this approach, however, the size of the prevention unit 110 inevitably increases, which affects heat conducting properties between the prevention unit 110 and the interior of the distal end section 10a.

In view of the above, in the present embodiment, the conducting section 170, which functions as an area having the lowest insulating properties, is provided in the mounting area portion 140b. Thereby, in the mounting area portion 140b, static electricity preferably flow to the conducting section 170 instead of over the base layer 141, the wiring portion 143, the connection part between the heating-side wiring portion 143a and the heating section 120, the connection part between the measuring-side wiring portion 143b and the measuring section 130, and parts of the heating section 120 and the measuring section 130 having low insulating properties. Thus, the static electricity flows to the GND from the conducting section 170 via the conductor-side wiring portion 143c and the lead line.

Thereby, the base layer 141, the wiring portion 143, the connection part between the heating-side wiring portion 143a and the heating section 120, the connection part between the measuring-side wiring portion 143b and the measuring section 130, the heating section 120, and the measuring section 130 are protected from static electricity.

Further, the conducting section 170 surrounds the heating section 120 and the measuring section 130 in the plane direction of the base layer 141, is in proximity to the heating section 120 and the measuring section 130, and is exposed to the cover member 145.

Accordingly, static electricity preferably and reliably flows to the conducting section 170. Thereby, the base layer 141, the wiring portion 143, the connection part between the heating-side wiring portion 143a and the heating section 120, the connection part between the measuring-side wiring portion 143b and the measuring section 130, the heating section 120, and the measuring section 130 are reliably protected from static electricity by the conducting section 170.

Further, the conducting section 170 is provided on the base layer 141. Accordingly, relative positions between the conducting section 170 and the heating section 120 and the measuring section 130 are determined with high precision.

Thereby, an improved protection effect against static electricity is achieved, compared to the state in which a protection mechanism against static electricity is provided separately from a heating unit as an exterior component, for example.

Thus, by providing the conducting section 170, a high resistance to static electricity is reliably secured.

[Effect]

As described above, in the present embodiment, static electricity preferably flows to the conducting section 170 in the mounting area portion 140b.

In the present embodiment, in particular, the conducting section 170 surrounds the heating section 120 and the measuring section 130 in the plane direction of the base layer 141, is in proximity to the heating section 120 and the measuring section 130, and is exposed to the cover member 145, as shown in FIG. 4A. Thereby, static electricity preferably and reliably flows to the conducting section 170 in the mounting area portion 140b.

Further, in the present embodiment, as shown in FIGS. 4C, 4D, 5A, and 5B, the conducting section 170 is provided on the base layer 141, and relative positions of the conducting section 170, the heating section 120, and the measuring section 130 are determined with high precision. Accordingly, an increased protection effect against static electricity is achieved.

It is thus possible to reliably secure a high resistance to static electricity in the present embodiment.

Further, in the present embodiment, since the conducting section 170 is provided, it is not necessary to increase the creepage distance in which static electricity enters, and to cause the first sealing member 171 to firmly perform the sealing. It is thus possible in the present embodiment to reduce the size of the prevention unit 110, and to prevent heat conducting properties between the prevention unit 110 and the interior of the distal end section 10a from being affected.

Further, in the present embodiment, since the first sealing member 171 is provided as shown in FIGS. 5A and 5B, it is possible to prevent entry of static electricity into the connection part between the heating-side wiring portion 143a and the heating section 120, the connection part between the measuring-side wiring portion 143b and the measuring section 130, the heating section 120, and the measuring section 130. It is thereby possible to reliably secure high resistance to static electricity in the present embodiment by providing the conducting section 170 and the first sealing member 171.

The conducting section 170 may include projections such as stud bumps in the mounting area portion 140b. The projections are formed in the form of dots, for example, and are provided in the vicinity of four corners of the heating section 120 and in the vicinity of four corners of the measuring section 130, for example. It is thus possible in the present embodiment to reliably let static electricity flow to the conducting section 170 via the projections.

### Second Embodiment

[Configuration]

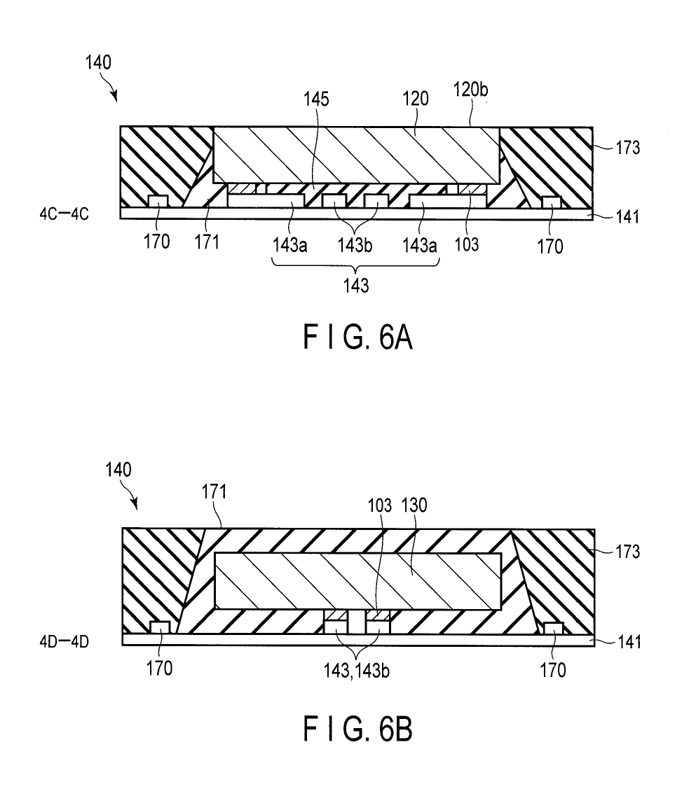

The present embodiment will be described with reference to FIGS. 6A and 6B. Only structures different from those of the first embodiment will be discussed below.

[Second Sealing Member 173]

A prevention unit 110 further includes a second sealing member 173 configured to seal a base layer 141, a conducting section 170, and a first sealing member 171 and having a volume resistivity lower than that of the first sealing member 171, as shown in FIGS. 6A and 6B.

For example, when the heating section 120 includes a heating chip 121, a ceramic substrate 123, a metal resistance 125, and a pad 127, the ceramic substrate 123 has insulating properties. Accordingly, in consideration of heat conducting properties, the second sealing member 173 seals the base layer 141, the conducting section 170, the first sealing member 171, and the heating section 120, such that a bonding surface 120b of the heating section 120 bonded to a lens frame 40 is exposed, as shown in FIG. 6A.

When a measuring section 130 is formed of a bulk, for example, the second sealing member 173 seals the base layer 141, the conducting section 170, and the first sealing member 171, such that an upper surface of the first sealing member 171 is exposed, as shown in FIG. 6B.

In view of the volume resistances, the first sealing member 171 is formed of an epoxy-based resin, for example, and the second sealing member 173 is formed of an epoxy-based resin into which carbon is added, for example.

[Action]

The volume resistance of the second sealing member 173 is lower than the volume resistance of the first sealing member 171. Accordingly, in the present embodiment, static electricity preferably flows to the second sealing member 173 instead of over the first sealing member 171. After that, the static electricity flows from the second sealing member 173 to the conducting section 170.

[Effect]

In the present embodiment, the second sealing member 173 is provided. Thereby, in the present embodiment, the conducting section 170 reliably obtains desired insulating resistance to the outside.

Further, in the present embodiment, the volume resistance of the second sealing member 173 is lower than the volume resistance of the first sealing member 171. Thereby, in the present embodiment, priorities in insulating properties can be assigned to the area to be protected from static electricity, such as a connection part between the wiring portion 143 and the heating section 120 and the measuring section 130, the heating section 120 and the measuring section 130, and the area through which static electricity flows, such as the conducting section 170, by the first sealing member 171 and the second sealing member 173. It is thereby possible in the present embodiment to reliably and stably secure high resistance to static electricity, and to provide the conducting section 170 reliably having desired insulating resistance against the outside.

Further, in the present embodiment, since the second sealing member 173 is provided, the conducting section 170 reliably achieves desired insulating resistance, compared to a configuration in which a space is provided between the conducting section 170 and an endoscope structure. Further, in the present embodiment, since the second sealing member 173 is provided, it is possible to reliably make the potential of the conducting section 170 close to the potential of the GND, compared to a configuration in which a resistive element is provided between a conducting section 170 and a GND.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope fogging prevention unit comprising:
a distal end section including a cylindrical member, in an interior of which at least an optical member is provided;
a heating section provided in the interior of the distal end section and configured to heat at least the optical member;
a measuring section provided in the interior of the distal end section and configured to measure a temperature of the interior of the distal end section;
a wiring substrate section including a mounting area portion in which the heating section and the measuring section are mounted, and a wiring area portion in which a wiring member connected to the heating section and the measuring section is provided; and
a conducting section provided in the mounting area portion, having the lowest insulating properties against the outside in the mounting area portion, and connected to a ground of a system different from a system of the wiring member of the heating section and the measuring section.

2. The endoscope fogging prevention unit according to claim 1, wherein the conducting section is provided in the periphery of the heating section and the measuring section.

3. The endoscope fogging prevention unit according to claim 1, wherein the conducting section is provided so as to surround the heating section and the measuring section in a plane direction of the mounting area portion.

4. The endoscope fogging prevention unit according to claim 1, further comprising:
a first sealing member provided at least at a connection part between the wiring member and the heating section and the measuring section such that the conducting section is exposed, configured to seal the connection part, and having insulating properties.

5. The endoscope fogging prevention unit according to claim 4, further comprising:
a second sealing member configured to seal the conducting section, and having a volume resistivity lower than a volume resistivity of the first sealing member.

6. An endoscope comprising the endoscope fogging prevention unit according to claims 1.
